(19) United States
(12) Patent Application Publication
(10) Pub. No.: US 2024/0047016 A1
(43) Pub. Date: Feb. 8, 2024

(54) DEVICE FOR ANALYZING MATERIAL FROM UNKNOWN SAMPLE BASED ON ARTIFICIAL INTELLIGENCE AND METHOD OF USING THE SAME

(71) Applicant: DAEJIN ADVANCED MATERIALS INC., Gyeonggi-do (KR)

(72) Inventor: Gwan Yeong KIM, Gyeonggi-do (KR)

(21) Appl. No.: 17/760,503

(22) PCT Filed: Dec. 24, 2021
(86) PCT No.: PCT/KR2021/019801

§ 371 Date: Mar. 15, 2022

(30) Foreign Application Priority Data
Dec. 1, 2021 (KR) 10-2021-0170098

(51) Int. Cl. G16C 20/30; G16C 20/90; G16C 20/70

(52) U.S. Cl. CPC G16C 20/30; G16C 20/90; G16C 20/70

(57) ABSTRACT

The present invention relates to an artificial intelligence-based device for analyzing a material from an unknown sample, which includes a material analysis unit configured to acquire at least three pieces of characteristic analysis data from an unknown sample; a sample classification unit configured to compare the acquired characteristic analysis data with data included in a property database to determine a similarity score and a confidence score, and to store the scores in a sample database; a comprehensive analysis unit configured to learn and verify the characteristic analysis data on the basis of the similarity score and the confidence score according to a predetermined condition to analyze a material; and a data output unit configured to store a material analysis result in an output database, and a method employing the device.

### TECHNICAL FIELD

This application claims priority to and the benefit of Korean Patent Application No. 10-2021-0170098, filed on Dec. 1, 2021, the disclosure of which is incorporated herein by reference in its entirety.

The present invention relates to an artificial intelligence (AI)-based device for analyzing a material from an unknown sample.

### BACKGROUND ART

In the case of using a recycled resin of which a chemical composition is difficult to know, it is difficult to know what kinds of materials are included. When it is possible to accurately estimate what kinds of materials the recycled resin is made of, the materials may be used for reproducing a resin of a new composition.

To analyze the materials, a method of analyzing the type or content of a material through a mass spectrometer may be used, but the method has the problem of low accuracy. Further, even when two types of spectrometers are used, errors frequently occur in determining the composition of an unknown material.

Therefore, it is necessary to develop a device for estimating the materials of a recycled resin with high accuracy or high reliability.

### SUMMARY

### Technical Problem

The present invention is directed to providing a device for recommending the composition of a composite material or a production process of a composite material for a target property by utilizing artificial intelligence (AI).

The present invention is also directed to providing a method of recommending the composition of a composite material or a production process of a composite material for a target property by utilizing AI.

### Technical Solution

One aspect of the present invention provides an artificial intelligence (AI)-based device for analyzing a material from an unknown sample, the device including a material analysis unit configured to acquire at least three pieces of characteristic analysis data from an unknown sample; a sample classification unit configured to compare the acquired characteristic analysis data with data included in a property database to determine a similarity score and a confidence score, and to store the scores in a sample database; a comprehensive analysis unit configured to perform learning and verification on the basis of the similarity score and the confidence score according to a predetermined condition to analyze a material; and a material output unit configured to store the material analysis result in an output database.

Another aspect of the present invention provides an AI-based method of analyzing a material from an unknown sample, which uses the device and is implemented by a computer, the method including: acquiring at least three pieces of characteristic analysis data from an unknown sample; comparing the acquired characteristic analysis data with data included in a property database and determining a similarity score and a confidence score; and performing learning and verification on the basis of the similarity score and the confidence score according to a predetermined condition to analyze a material and storing the analysis result in an output database.

In an embodiment of the present invention, the material output unit may further include an output adjustment unit configured to determine an output form according to the number of estimated samples, a similarity score, or a confidence score set by a user.

In an embodiment of the present invention, the output database may include a first material output database configured to store the material analysis result of the comprehensive analysis unit; and a second material output database configured to store the material result determined by the output adjustment unit.

In an embodiment of the present invention, the comprehensive analysis unit may give a weight to a similarity score and a confidence score of each piece of the characteristic analysis data and analyze the material.

In an embodiment of the present invention, the characteristic analysis data may be chemical structure data, optical property data, mechanical property data, electrical property data, thermal property data, or magnetic property data.

The chemical structure data may be at least one of nuclear magnetic resonance (NMR) data, X-ray photoelectron spectroscopy (XPS) data, energy dispersive X-ray spectroscopy (EDS) data, elemental analysis data, gel permeation chromatography (GPC) data, and cyclic voltammetry (CV) data, and weights may be preset for the pieces of data.

The optical property data may be at least one of ultraviolet/visible spectroscopy (UV-Vis) data, Fourier-transform infrared spectroscopy (FTIR) data, Raman spectroscopy data, X-ray spectroscopy (XRF) data, gamma spectroscopy data, and ellipsometry data, and weights may be preset for the pieces of data.

The mechanical property data may be at least one of universal testing machine (UTM) data, Izod impact strength data, and dynamic mechanical test analysis test data, and weights may be preset for the pieces of data. The electrical property data may be at least one of electrical conductivity data, dielectric constant hysteresis data, and ultraviolet photoelectron spectroscopy (UPS) data, and weights between the pieces of data may be preset.

The thermal property data may be at least one of differential scanning calorimetry (DSC) data, thermogravimetric analysis (TGA) data, and thermal conductivity-T plot data, and weights may be preset for the pieces of data.

The magnetic property data may be at least one of electron spin resonance (ESR) data and magnetoresistance analysis data, and weight between the pieces of data may be preset.

In an embodiment of the present invention, the property database may be connected to an external database through an Internet network or a self-established network.

Another aspect of the present invention provides an AI-based device for recommending a composition-process of a composite material, which includes an AI-based device for analyzing a material from an unknown sample, the AI-based device including: a data collection unit configured to collect composition-process condition data for a target property input by a user and store the collected condition data in a collection database; an input grade classification unit configured to classify the collected condition data into different input grades according to an input grade determination factor; a training data supply unit configured to store the condition data classified into the input grades in a training database and input condition data of a preset high grade in the training database; a model generation unit configured to learn and verify the data input from the training data supply unit and generate a composition-process model; and a material-process data output unit configured to derive one or more composition-process conditions for the target property and store the derived composition-process conditions in a material-process output database.

In an embodiment of the present invention, the device may further include a reasoning database configured to extract reasoning condition data for the target property and send the extracted reasoning condition data to the model generation unit in order to compare and verify the reasoning condition data with the condition data supplied from the training data supply unit.

In an embodiment of the present invention, the device may further include a model variation unit configured to vary the model generated by the model generation unit according to a variation condition input by the user.

In an embodiment of the present invention, the model variation unit may include a relationship comparison unit; configured to compare a relationship between a variant material condition or variant synthesis method input by the user and a material condition or synthesis method input by the training data supply unit and a condition variation unit; configured to replace one or more factors of a material combination condition and synthesis process condition generated by the model generation unit with other factors or vary the material combination condition and synthesis process condition generated by the model generation unit to include one or more additional factors.

In an embodiment of the present invention, the relationship comparison unit may compare and verify a physical relationship additionally input by the user.

In an embodiment of the present invention, the material-process data output unit may include a first material-process output database configured to store one or more composition-process conditions derived by a model unit; an output grade classification unit configured to classify the resulting conditions into separate grades according to an output grade determination factor; and a second material-process output database configured to store an output grade according to a composition-process condition, which satisfies the output grade determination factor input by the user.

In an embodiment of the present invention, the output grade determination factors of the output grade classification unit may be one or more of a unit price, a yield, a processing time, a preferred process, and existing experience.

In an embodiment of the present invention, the input grade determination factor of the input grade classification unit may be one or more of a data type, a data characteristic, a property weight, and an error range.

In an embodiment of the present invention, the input grade classification unit may include a paper or report data characteristic grade classification unit configured to give a weight to each of qualitative grades of academic journals, each of qualitative grades of papers or reports, and/or each of publication years of the papers or reports and give a characteristic grade according to the weight; a laboratory data characteristic grade classification unit and factory production data characteristic grade classification unit configured to give a characteristic grade according to a user input value and/or a similar value to a repeatedly input value; a patent data characteristic grade classification unit configured to give a characteristic grade according to the number of family countries, a patent application year, and/or the number of citations; and a weight giving unit configured to determine an input grade in consideration of weights and error ranges according to the order of priority and/or a collection path among a plurality of property values input by the user.

Another aspect of the present invention provides an AI-based method which includes a device for analyzing a material from an unknown sample on the basis of artificial intelligence, of recommending a composition-process of a composite material, which is performed by an AI-based device for recommending a composition-process of a composite material, implemented by a computer, the method including: collecting composition-process condition data for a target property input by a user; classifying the collected condition data into different input grades according to an input grade determination factor; storing the condition data classified into the input grades in a training database and inputting condition data of a predetermined high grade to a training data supply unit; learning and verifying the data input from the training data supply unit and generating a composition-process model; and deriving, by the model, one or more composition-process conditions for the target property and storing, by a material-process data output unit, the derived material-process conditions in an output database.

In an embodiment of the present invention, the generating of the composition-process model may further include comparing and verifying data for the target property derived from a reasoning database stored in a data reasoning unit with the data supplied from the training data supply unit.

In an embodiment of the present invention, the generating of the composition-process model may further include varying the model according to a variation condition input by the user.

In an embodiment of the present invention, the model variation operation may include comparing a relationship between a variant material condition or variant synthesis method input by the user and a material condition or synthesis method input by the training data supply unit; and deriving a modification condition to replace one or more factors of a material composition condition and synthesis process condition generated by a model generation unit with other factor or include one or more additional factors.

In an embodiment of the present invention, the comparing of the relationship may include comparing and verifying a physical relationship additionally input by the user.

In an embodiment of the present invention, the storing in the material-process output database may include storing one or more composition-process conditions generated by a model unit in a first material-process output database; classifying, by an output grade classification unit, the conditions into separate grades according to an output grade determination factor; and storing an output grade according to a composition-process condition satisfying the output grade determination factor input by the user in a second material-process output database.

In an embodiment of the present invention, the storing in the material-process output database, the output grade determination factor may be one or more selected from among a unit price, a yield, a processing time, a preferred process, and preexistence experience.

In an embodiment of the present invention, in the classifying of the collected condition data into the different input grades, the input grade determination factor may be one or more selected from a data type, a data characteristic, a property weight, and an error range.

In an embodiment of the present invention, in the classifying of the collected condition data into the different input grades, the data type may be one or more of paper or report data, laboratory data, factory production data, and patent data.

In an embodiment of the present invention, in the classifying of the collected condition data into the different input grades, the paper or report data may give a weight to each of qualitative grades of academic journals, each of qualitative grades of papers or reports, and/or each of publication years of the papers or reports and give a characteristic grade according to the weight, the laboratory data and the factory production data may give a characteristic grade according to a user input value and/or a similar value to a repeatedly input value, and the patent data may give a characteristic grade according to the number of family countries, a patent application year, and/or the number of citations.

In the classifying of the collected condition data into the different input grades according to an embodiment of the present invention, a weight determining an input grade may be given by considering property weights and error ranges according to the order of priority and/or a collection path among a plurality of properties input by the user, and giving a weight for each characteristic grade.

In an embodiment of the present invention, when the user gives data as a high reliability value or a repeatedly input value, the laboratory data and the factory production data may be set to a high value, a grade of data having a similar value to the high value may be upgraded, a grade may be lowered or a difference between grades may be increased when there is a greater difference, and a higher grade may be given to the patent data when there are a greater number of family countries or a patent application has been filed more lately.

In an embodiment of the present invention, the classifying of the collected condition data into the different input grades may include giving a weight determining an input grade by considering property weights and error ranges according to the order of priority among a plurality of properties input by the user, and giving a weight for each characteristic grade.

### Advantageous Effects

A device and method according to an embodiment of present invention generate a composition and production process of a composite material by utilizing artificial intelligence (AI), and thus it is possible to derive an optimal composition and process. Accordingly, the device and method can show high accuracy and reliability, and it is possible to save costs and time for deriving an optimal composition and process through experiments.

A device and method according to an embodiment of present invention perform machine learning with online data, such as paper data and patent data, and thus it is possible to derive an optimal composition and process for the latest trend.

A device and method according to an embodiment of present invention has an advantage in that a user can select one of a plurality of compositions and process conditions that satisfy a specific property according to a desired determination factor.

### BRIEF DESCRIPTION OF THE DRAWINGS

FIG. 1 is a conceptual diagram of determining a composition-process method after analyzing a material from an unknown sample according to an embodiment of the present invention.

FIGS. 2, 3 and 4 are block diagrams of a device according to an embodiment of the present invention.

FIG. 5 illustrates an arrangement example of a sample database according to an embodiment of the present invention.

FIG. 6 is a block diagram of a server according to an embodiment of the present invention.

FIG. 7 illustrates an arrangement example of a second material output database according to an embodiment of the present invention.

FIG. 8 is a block diagram of a composition-process recommendation device according to an embodiment of the present invention.

FIG. 9 is a block diagram of a server according to an embodiment of the present invention.

FIG. 10 is a block diagram of a server according to an embodiment of the present invention.

FIG. 11 is a block diagram of a model variation unit according to an embodiment of the present invention.

FIG. 12 is a block diagram of a material-process data output unit according to an embodiment of the present invention.

FIGS. 13 and 14 illustrate output grades determined according to output grade determination factors according to an embodiment of the present invention.

FIG. 15 is a block diagram of an input grade classification unit according to an embodiment of the present invention.

FIG. 16 illustrates grades determined by an input grade classification unit according to an embodiment of the present invention.

FIG. 17 is a flowchart illustrating an operating method of a device according to an embodiment of the present invention.

FIG. 18 is a flowchart illustrating an operating method of a device according to an embodiment of the present invention.

FIG. 19 is a flowchart illustrating a model variation operation method of a device according to an embodiment of the present invention.

### DETAILED DESCRIPTION

Hereinafter, the present invention will be described in detail.

The following specific functional descriptions are exemplified for the purpose of describing embodiments according to the concept of the present invention. Embodiments of the present invention can be implemented in various forms, and the present invention is not to be construed as being limited to the embodiments described herein.

Embodiments according to the concept of the present invention can de diversely modified and have various forms, and thus specific embodiments will be described in detail herein. However, this is not intended to limit embodiments according to the concept of the present invention to the specific embodiments set forth herein, and the specific embodiments should be construed as covering all modifications, equivalents, and alternatives falling within the spirit and technical scope of the present invention.

Terms used herein are merely used to describe particular embodiments and are not intended to limit the scope of the present invention. Singular expressions include plural expressions unless the context clearly indicates otherwise.

Unless otherwise defined, all terms including technical and scientific terms used herein have the same meaning as commonly understood by those of ordinary skill in the art. Terms, such as those defined in commonly used dictionaries, should be interpreted as having a meaning that is consistent with their meaning in the context of the relevant art and should not be interpreted in an idealized or overly formal sense unless expressly so defined herein.

In the specification, it is to be understood that the term “comprise,” “include,” or “have” specifies the presence of stated features, integers, steps, operations, components, parts, or combinations thereof but does not preclude the presence or addition of one or more other features, integers, steps, operations, components, parts, or combinations thereof.

When a component is referred to as being “connected,” “transmitting,” “sending,” “receiving,” or “delivering” something to another component, the component may be directly coupled or transmit, send, receive, or deliver something to the other component, and also a third component may be present between the two components such that the component is indirectly connected or transmits, sends, receives, or delivers something to the other component.

As used herein, the term “unit” means a software or hardware component and performs a specific function. However, the term “unit” is not limited to software or hardware. A “unit” may be configured to be in an addressable storage medium or to operate one or more processors. Accordingly, for example, “units” include components, such as software components, object-oriented software components, class components, and task components, processes, functions, attributes, procedures, subroutines, segments of program code, drivers, firmware, microcode, circuits, data, databases, data structures, tables, arrays, and variable. A function provided by components and “units” may be associated with a smaller number of components and “units” or may be subdivided into additional components and “units.”

According to an embodiment of the present invention, a “unit” may be implemented as a processor and a memory. The term “processor” should be interpreted in a broad meaning to encompass a general-purpose processor, a central processing unit (CPU), a microprocessor, a digital signal processor (DSP), a controller, a microcontroller, a state machine, etc. Under some circumstances, a “processor” may refer to an application-specific integrated circuit (ASIC), a programmable logic device (PLD), a field-programmable gate array (FPGA), etc. The term “processor” may refer to a combination of processing devices, for example, a combination of a DSP and a microprocessor, a combination of a plurality of microprocessors, a combination of one or more microprocessors combined with a DSP core, or a combination of any other such configurations.

As used herein, the term “memory” should be interpreted in a broad meaning to encompass any electronic component for storing electronic information. The term “memory” may refer to various types of processor-readable media such as a random access memory (RAM), a read-only memory (ROM), a non-volatile random access memory (NVRAM), programmable read-only memory (PROM), an erasable programmable read-only memory (EPROM), an electrically erasable PROM (EEPROM), a flash memory, a magnetic or optical data storage, and registers. While a processor may read information from a memory and/or record information in a memory, a memory is referred to as being in electronic communication with a processor. A memory integrated with a processor is in electronic communication with the processor.

As used herein, “composition” means kinds and amounts of components constituting a material and includes not only components directly constituting the material but also all materials used in the process of producing the material, for example, a cross-linking agent, a catalyst, and a foaming agent. Also, a “property” can be not only a chemical characteristic but also any of physical, mechanical, or electronical characteristic.

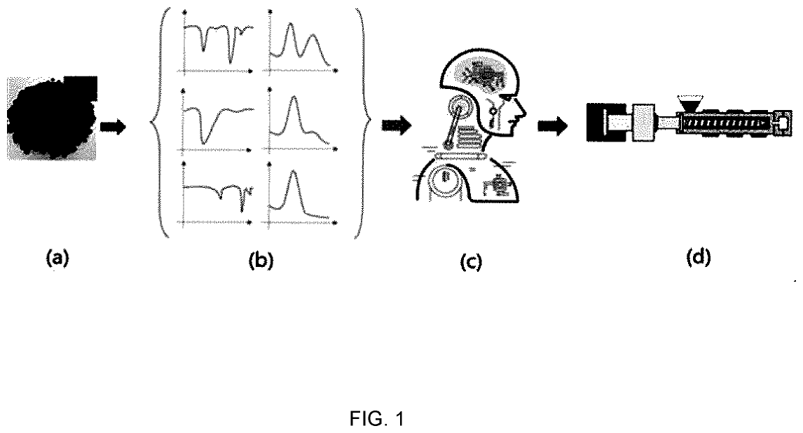

FIG. 1 is a conceptual diagram of determining a composition and process method after analyzing a material from an unknown sample according to an embodiment of the present invention.

Referring to FIG. 1, a material analysis device may be used for estimating a material of an unknown sample, and a composition-process device may be used for receiving a composition-process for a desired target property and easily producing a desired product. FIG. 1A shows an unknown recycled resin, FIG. 1B shows a property database, and FIG. 1C shows an operation of analyzing a sample using property analysis data and sample characteristic data on the basis of an artificial intelligence algorithm and estimating a material. FIG. 1D shows an operation of recommending a manufacturing process for an optimal composition-process for obtaining a target property wanted by a user, and then, a desired product may be produced using a recommended device and/or method.

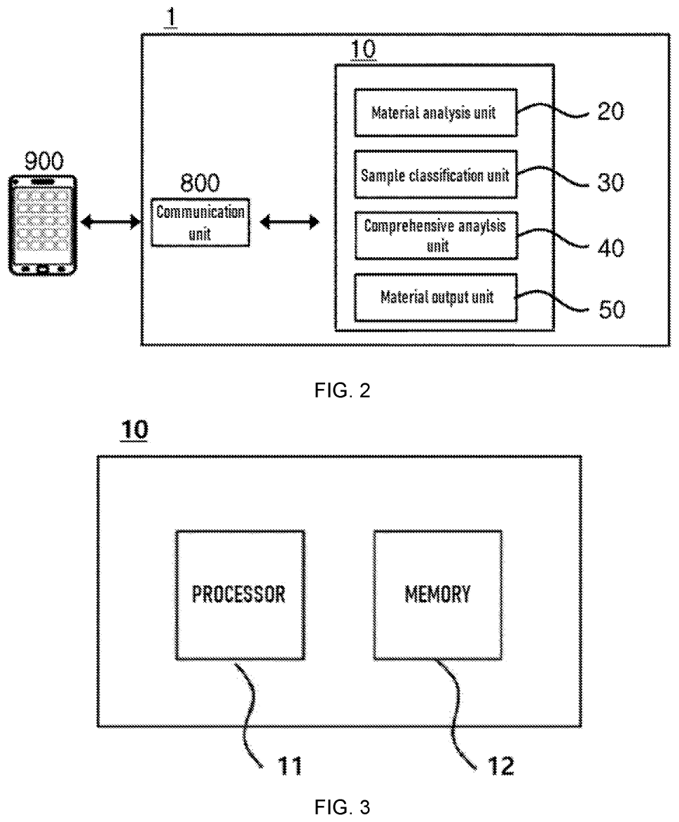

FIGS. 2 to 4 are block diagrams of a device according to an embodiment of the present invention.

A user terminal 900 may be a portable mobile terminal that ensures portability and mobility. The terminal may encompass any type of handheld wireless communication device such as a personal communication system (PCS), a global system for mobile communications (GSM), a personal digital cellular (PDC), a personal handyphone system (PHS), a personal digital assistant (PDA), a wireless broadband and Internet (WiBro) terminal, a smartphone, or a tablet personal computer (PC). In particular, in the present invention, a user terminal is an intelligent device obtained by adding computer-support functions, such as an Internet communication function and an information retrieval function, to a portable device and may be a smartphone on which a plurality of desired applications may be installed and executed by a user. Also, the user terminal 900 may be implemented as a general-use computer such as a desktop or a laptop.

A communication unit 800 may support not only data transmission and reception between components in an AI model generation device of the present invention but also establishment of a wired or wireless communication channel with the user terminal 900 to be described below and communication through the established communication channel. The communication unit 800 may include at least one communication processor that supports wired communication or wireless communication. The communication unit 800 may include a wireless communication module (e.g., a cellular communication module, a short-range wireless communication module, or a global navigation satellite system (GNSS) communication module) or a wired communication module (e.g., a local area network (LAN) communication module or a power line communication module).

The wireless communication may include cellular communication that employs at least one of, for example, Long Term Evolution (LTE), LTE advance (LTE-A), code division multiple access (CDMA), wideband CDMA (WCDMA), a universal mobile telecommunications system (UMTS), a wireless broadband (WiBro), or a GSM. According to an embodiment, the wireless communication may include at least one of, for example, wireless fidelity (WiFi), Bluetooth, Bluetooth Low Energy (BLE), ZigBee, near field communication (NFC), magnetic secure transmission, a radio frequency (RF), and a body area network (BAN). According to an embodiment, the wireless communication may include a GNSS. The GNSS may be, for example, a global positioning system (GPS), a global navigation satellite system (Glonass), a BeiDou navigation satellite system (hereinafter “BeiDou”), or Galileo, a European global satellite-based navigation system. In this specification, “GPS” and “GNSS” may be interchangeably used below. The wired communication may include at least one of, for example, a universal serial bus (USB), a high definition multimedia interface (HDMI), recommended standard 232 (RS-232), power line communication, and a plain old telephone service (POTS). A network may include at least one of telecommunication networks, for example, a computer network (e.g., a LAN or a wide area network (WAN)), the Internet, and a telephone network.

Referring to FIG. 3, the server 10 may include a processor 11 and a memory 12. The data collection unit 100 may include a processor and a memory, and the processor may execute a command stored in the memory. The input grade classification unit 200, the training data supply unit 300, the model generation unit 400, and the material-process data output unit 500 may also include the processor 11 and the memory 12. The user terminal 900 may also include a processor and a memory.

Referring to FIG. 2, the device includes a material analysis unit 20 for acquiring at least three pieces of characteristic analysis data from an unknown sample. In this case, all of three or more, four or more, five or more, and six or more pieces of characteristic data may be used. The number of pieces of data values that are efficient in terms of similarity and confidence may be selected and used.

According to an embodiment of the present invention, the characteristic analysis data may be chemical structure data, optical property data, mechanical property data, electrical property data, thermal property data, or magnetic property data.

The chemical structure data may be at least one of nuclear magnetic resonance (NMR) data, X-ray photoelectron spectroscopy (XPS) data, energy dispersive X-ray spectroscopy (EDS) data, elemental analysis data, gel permeation chromatography (GPC) data, and cyclic voltammetry (CV) data, and weights may be preset for the pieces of data.

The optical property data may be at least one of ultraviolet/visible spectroscopy (UV-Vis) data, Fourier-transform infrared spectroscopy (FTIR) data, Raman spectroscopy data, X-ray spectroscopy (XRF) data, gamma spectroscopy data, and ellipsometry data, and weights may be preset for the pieces of data.

The mechanical property data may be at least one of universal testing machine (UTM) data, Izod impact strength data, and dynamic mechanical test analysis test data, and weights may be preset for the pieces of data. The electrical property data may be at least one of electrical conductivity data, dielectric constant hysteresis data, and ultraviolet photoelectron spectroscopy (UPS) data, and weights may be preset for the pieces of data.

The thermal property data may be at least one of differential scanning calorimetry (DSC) data, thermogravimetric analysis (TGA) data, and thermal conductivity-T plot data, and weights may be preset for the pieces of data.

The magnetic property data may be at least one of electron spin resonance (ESR) data and magnetoresistance analysis data, and weights may be preset for the pieces of data.

The device includes a sample classification unit 30 for comparing the acquired characteristic analysis data with data included in a property database 61 to determine a similarity score and a confidence score, and storing the scores in a sample database 31. The property database 61 may be connected to an external database through an Internet network or a self-established network.

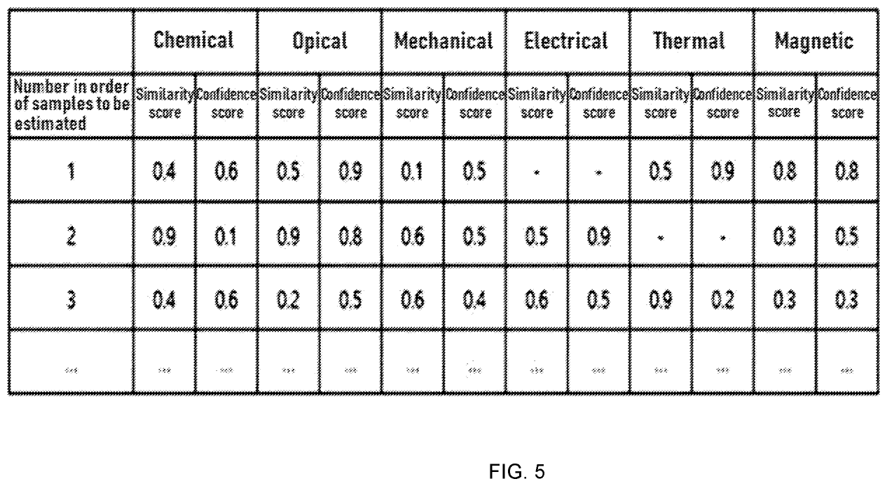

FIG. 5 illustrates an arrangement example of the sample database 31 according to an embodiment of the present invention. In the sample database 31, confidence scores, which represent how certain output values are, may be arranged together with similarity scores.

The sample classification unit 30 and the comprehensive analysis unit 40 may determine and analyze a similarity score and a confidence score using an artificial intelligence algorithm. Specifically, the artificial intelligence algorithm indicates multiple linear regression (MLR), a support vector machine (SVM), a k-nearest neighbor (KNN), a deep learning, generic algorithm (GA), boosted trees, a generative adversarial network (GAN), an artificial neural network (ANN), Ensemble, etc., and any one or more selected from among the algorithms may be used for determining a similarity score and a confidence score. Alternatively, a learning-based artificial intelligence algorithm may be used for determining a similarity score and a confidence score. The algorithm may include not only MLR, a deep neural network (DNN), a convolutional neural network (CNN), an SVM, a decision tree, boosted trees, a KNN, logistic regression, a GAN, an ANN, and Ensemble but also pre-published classifiers employing the algorithms. The classifiers may include commercialized classifiers such as a random forest classifier, an extreme gradient boosted, a tensorflow deep learning classifier, etc. In other words, there is no limitation on the plurality of artificial intelligence algorithm, and the classifiers that have been modeled already may be included in the artificial intelligence algorithms.

The device may include the comprehensive analysis unit 40 for performing learning and verification on the basis of the similarity score and the confidence score according to a predetermined condition to analyze a material. According to an embodiment of the present invention, the comprehensive analysis unit 40 may give a weight to a similarity score and a confidence score to each piece of the characteristic analysis data and analyze the material.

A weight for setting how greatly a similarity score or confidence score of a certain characteristic will be considered may be preset by the user. Also, the user may preset a specific characteristic so that only a certain level or more of similarity score or confidence score may be taken into consideration. For example, an electrical property may be used in comprehensive analysis only when the electrical property has a similarity score of 0.8 or more and a confidence score of 0.9 or more.

The device includes a material output unit 50 for storing the material analysis result in a material output database 51. According to an embodiment of the present invention, as a material data output unit, the material output unit 50 may additionally include a material output adjustment unit 52 that determines an output form according to the number of estimated samples, a similarity score, or a confidence score set by the user.

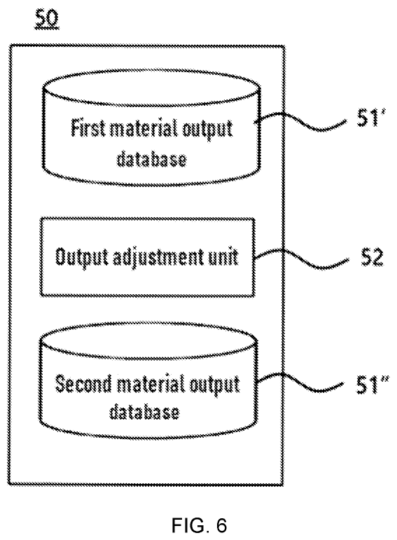

FIG. 6 is a block diagram of a server according to an embodiment of the present invention.

Referring to FIG. 6, as a material output database, the output database 51 may include a first material output database 51′ for storing the material analysis result of the comprehensive analysis unit; and a second material output database 51″ for storing the material result determined by the output adjustment unit 52.

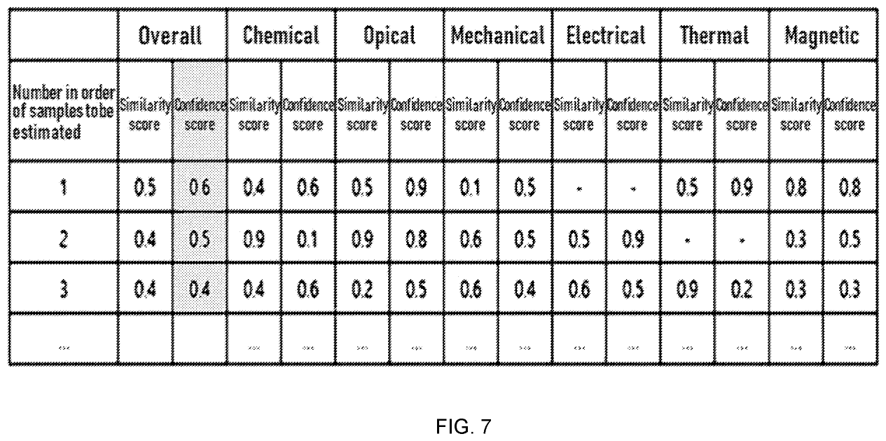

FIG. 7 illustrates an arrangement example of the second material output database 51″ according to an embodiment of the present invention. The output adjustment unit 52 may determine a final output form according to a sample type, a similarity score, and a confidence score stored in a first-order classification database and store data in the second output database 51″ according to a criterion preset by the user. The user may preset which one of similarity scores and confidence scores will be a base for arranging data or may preset the number of estimated materials to be output. The second material output database 51″ can output characteristic-specific similarity scores and confidence scores together with comprehensive similarity scores and confidence scores.

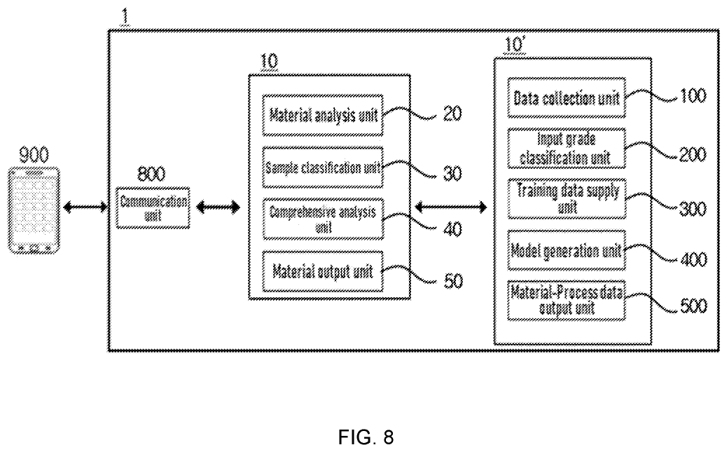

FIG. 8 is a block diagram of a composition-process recommendation device 1 according to an embodiment of the present invention.

Referring to FIG. 8, an artificial intelligence (AI)-based device for recommending a composition-process of a composite material may include a data collection unit 100 configured to collect composition-process condition data for a target property input by a user and store the collected condition data in a collection database 110; an input grade classification unit 200 configured to classify the collected condition data into different input grades according to input grade determination factors; a training data supply unit 300 configured to store the condition data classified into the input grades in a training database 310 and input condition data of a preset grade, specifically, a high grade or a specific grade, in the training database 310; a model generation unit 400 configured to learn and verify the data input from the training data supply unit and generate a composition-process model; and a material-process data output unit 500 configured to derive one or more composition-process conditions for the target property and store the derived composition-process conditions in a material-process output database 510.

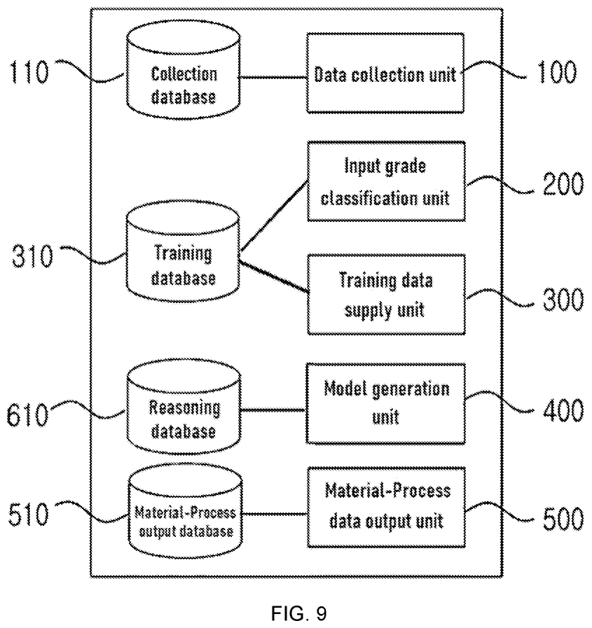

FIG. 9 is a block diagram of a server 10 according to an embodiment of the present invention.

Referring to FIG. 9, the data collection unit 100 may collect composition-process condition data for a target property input by a user and store the collected composition-process condition data in the collection database 110.

The training data supply unit 300 may store the condition data classified into input grades in the training database 310 and input preset condition data of a high grade in the training database to the model generation unit 400.

The model generation unit 400 may learn and verify the data input from the training data supply unit 300 and generate a composition-process model. Also, the model generation unit 400 may extract and receive reasoning condition data for the target property from a reasoning database 610 and may verity the model by comparing and verifying the condition data received from the training data supply unit 300.

The collected data is data about what property value a composite material has when it has a specific composition or is produced through a specific process.

The collected data may constitute a database in connection with a composition of the composite material, a content of each material, property data according to the contents, a process applied to each material, for example, compounding and injection, etc.

The composition of the composite material, the content of each material, the property data according to the contents, and the process applied to each material may be separated to extract data, or data may be extracted by analyzing the pieces of data in connection with each other.

Composition collection rules may include at least one of types of components, a possibility of combining components, a usage range of a component, and a generation interval of each component.

The types of components indicate which components are used in constituting a material. For example, three types of components A, B, and C may be used in constituting a specific material.

The possibility of combining components indicates a possibility of a combination such as specific components should be used together or exclusively used. For example, the possibility of combining components is a condition that the component B is not used when the component A is used, or the component C must be used together when the component A is used.

The usage range of a component may indicate the maximum usage or minimum usage of each component or the plurality of components. Here, the minimum value may be 0. For example, the maximum usage of the component A may be 100, the minimum usage of the component B may be 0.5 and the maximum usage of the component B may be 10. The usage range of a component may also be set for a plurality of components. For example, a minimum usage or maximum usage may be set for a total amount of the component A and the component B.

The generation interval of each component indicates the generation interval of each component when a plurality of virtual compositions are generated. For example, the component A may be generated in five units. Accordingly, the component A may be included in the plurality of virtual compositions as much as 100 units, 95 units, and 90 units. The generation interval of each component may be set for the whole usage range or a partial usage range of the corresponding component. For example, the generation interval of the component A may be set to five units for the whole usage range of 0 to 100, or the generation interval of the component A may be set to five units only for a partial section of 10 to 50 in the usage range and set to ten units for the remaining sections.

The target property input by the user may be one or more of an elastic modulus, a tensile strength, an impact strength, an electrical conductivity, a hardness, and a roughness, but is not limited thereto as long as a value can be recognized as a physical or chemical property of a composite resin.

The input grade classification unit 200 may train a machine learning model for performing a target task using the collected data.

The input grade classification unit 200 may train a machine learning model for performing a target task using the collected data.

At least one of the data collection unit 100, the input grade classification unit 200, and the training data supply unit 300 may be manufactured in the form of a hardware chip and mounted on an electronic device. At least one of the data collection unit 100, the input grade classification unit 200, and the training data supply unit 300 may be manufactured in the form of a dedicated hardware chip for AI or may be manufactured as a part of an existing general-use processor (e.g., a CPU or an application processor) or a dedicated graphics processor (e.g., a graphics processing unit (GPU)) and mounted on the various electronic devices described above.

At least one of the data collection unit 100, the input grade classification unit 200, and the training data supply unit 300 may be implemented as a software module. When at least one of the data collection unit 100, the input grade classification unit 200, and the training data supply unit 300 is implemented as a software module (or a program module including instructions), the software module may be stored in a memory or non-transitory computer-readable media. In this case, the at least one software module may be provided by an operating system (OS) or a certain application. Alternatively, some of the at least one software module may be provided by the OS, and remaining others may be provided by the certain application.

Label information may be allocated to each of a plurality of pieces of data. The label information may be information describing each of the plurality of pieces of data. The label information may be information to be derived by the target task. The label information may be acquired from a user input, a memory, or a result of the machine learning model.

An AI model generation algorithm of the model generation unit 400 which may indicate multiple linear regression (MLR), a support vector machine (SVM), a k-nearest neighbor (k-NN), deep learning, generic algorithm (GA), boosted trees, a generative adversarial network (GAN), an artificial neural network (ANN) or Ensemble, etc. and the model generation unit 400 may generate an AI model by applying any one or more selected from the above algorithms.

The AI algorithms may be a learning-based algorithm. The AI algorithms may include not only MLR, a deep neural network (DNN), a convolutional neural network (CNN), an SVM, a decision tree, a boosted tree, a k-NN, logistic regression, a GAN, an ANN, and Ensemble but also pre-published classifiers employing the algorithms. The classifiers may include commercialized classifiers such as a random forest classifier, an extreme gradient boosted, a tensorflow deep learning classifier, etc. In other words, there is no limitation on the plurality of AI algorithms, and the classifiers that have been modeled already may be included in the AI algorithms.

When property information requested by the user is input, the generated AI model may recommend composition information, process information, and composition-process information for providing the property.

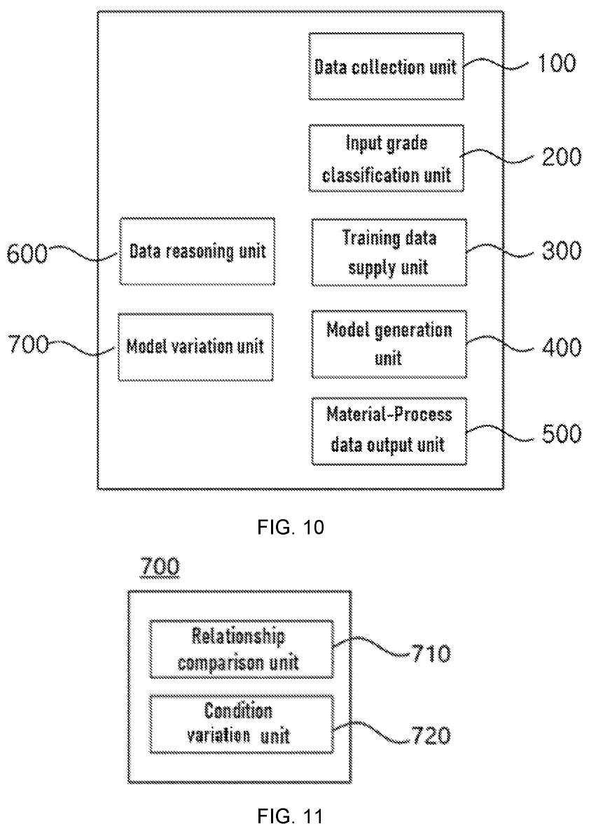

FIG. 10 is a block diagram of a server according to an embodiment of the present invention, and FIG. 11 is a block diagram of a model variation unit 700 according to an embodiment of the present invention.

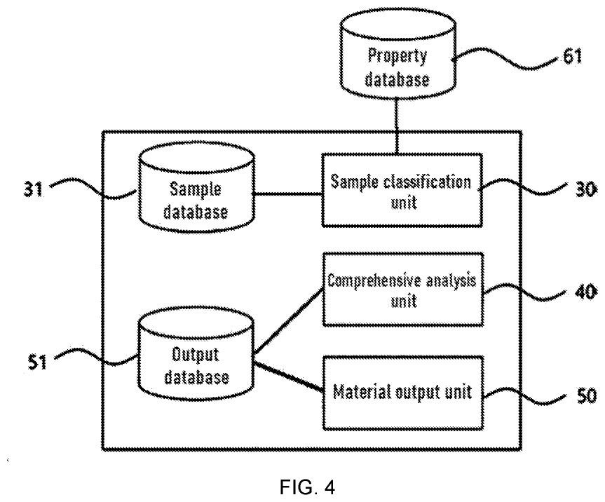

Referring to FIG. 4, the device may further include a data reasoning unit 600 that compares and verifies data derived for a target property from the reasoning database and data supplied from the training data supply unit. Also, the device may further include a model variation unit 700 that varies the model generated by the model generation unit 400 according to a variation condition input by the user. In addition, the device may further include the data reasoning unit 600 and the model variation unit 700.

The device may further include a model variation unit 700 that varies the model generated by the model generation unit 400 according to the variation condition input by the user.

Referring to FIG. 11, the model variation unit 700 may include a relationship comparison unit 710 that compares a relationship between a variant material condition or variant synthesis method input by the user and a material condition or synthesis method input by the training data supply unit 300; and a condition modification unit 720 that modifies the material combination condition and synthesis process condition to replace one or more factors of a material combination condition and synthesis process condition generated by the model generation unit 400 with other factors or include one or more additional factors.

In an embodiment of the present invention, the relationship comparison unit 710 may compare and verify a physical relationship additionally input by the user.

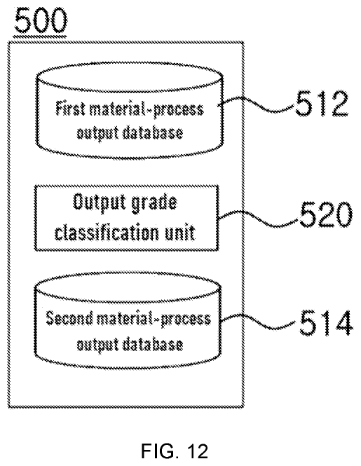

FIG. 12 is a block diagram of a material-process data output unit according to an embodiment of the present invention.

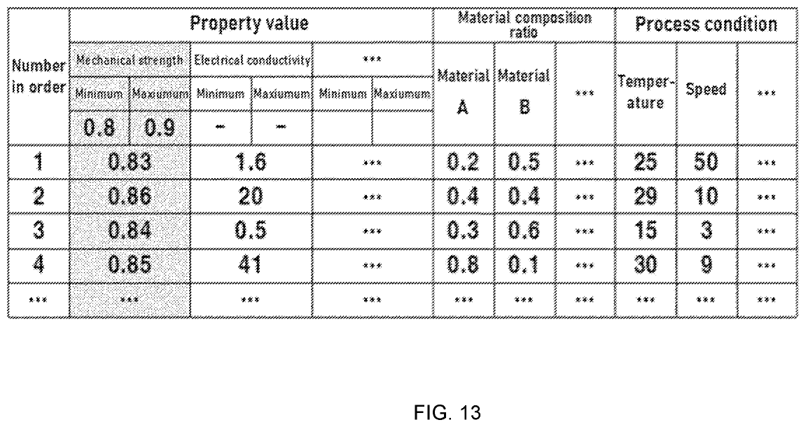

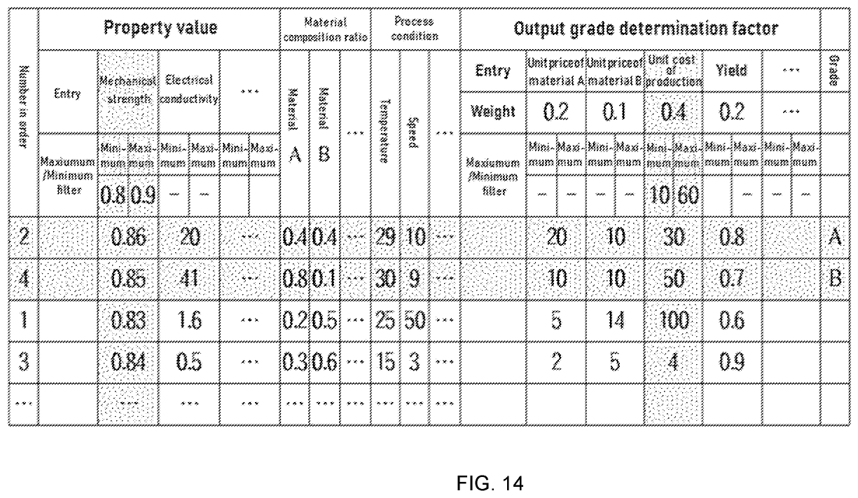

FIGS. 13 and 14 illustrate output grades determined depending according to output grade determination factors according to an embodiment of the present invention.

Referring to FIGS. 6 to 8, the material-process data output unit 500 may include a first material-process output database 512 that stores one or more composition-process conditions derived by a model unit; an output grade classification unit 520 that classifies the conditions into separate grades according to an output grade determination factor; and a second material-process output database 514 that stores an output grade according to a composition-process condition satisfying the output grade determination factor input by the user.

In an embodiment of the present invention, the output grade determination factor of the output grade classification unit 520 may be a unit price, a yield, a processing time, a preferred process, or preexistence experience. When the user inputs one or more of a unit price, a yield, a processing time, and a preferred process in advance, the input determination factors may be reclassified into grades in consideration of preexistence experience on the system in addition to the input determination factors and stored in the second material-process output database 514. For example, when the user inputs a unit price as an output grade determination factor and a yield has the largest amount of stored preexistence experience during a certain period (three months, one year, three years or etc.), the unit price may be reclassified into a first position, and the yield may be reclassified into a second position.

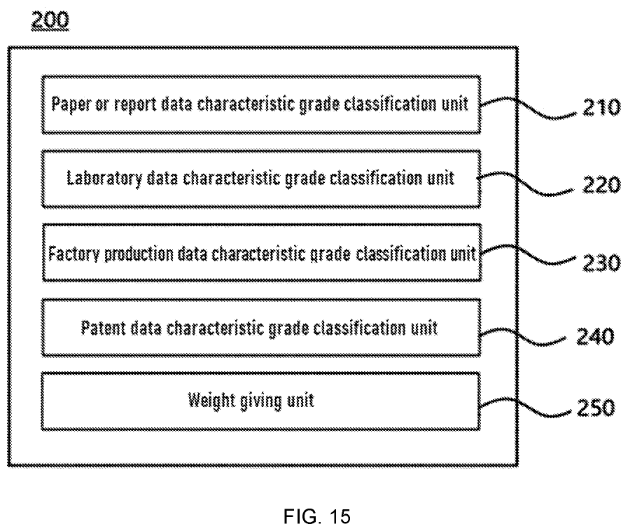

FIG. 15 is a block diagram of an input grade classification unit according to an embodiment of the present invention.

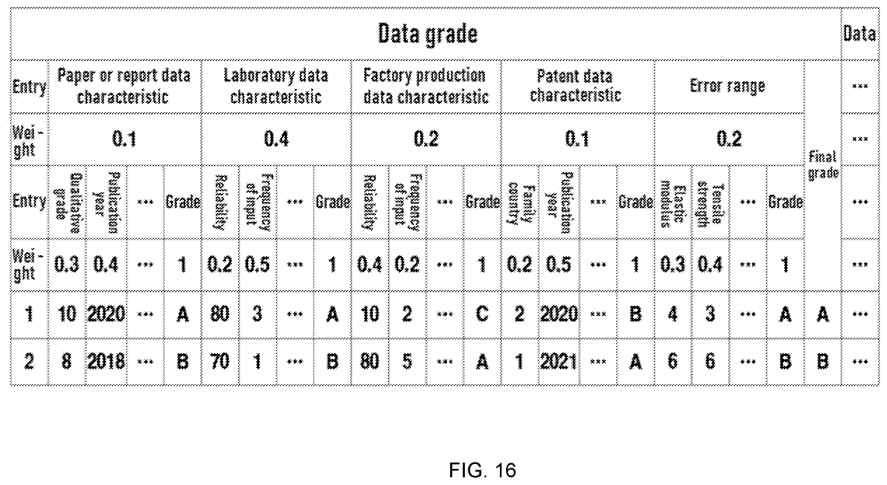

FIG. 16 illustrates grades determined by an input grade classification unit according to an embodiment of the present invention.

In an embodiment of the present invention, input grade determination factors of the input grade classification unit 200 may be one or more of a data type, a data characteristic, a property weight, and an error range.

Referring to FIGS. 9 and 10, the input grade classification unit 200 may include a paper or report data characteristic grade classification unit 210 that gives a weight to each of qualitative grades of papers or reports and/or each of publication years of the papers or reports and gives a characteristic grade according to the weight; a laboratory data characteristic grade classification unit 220 and a factory production data characteristic grade classification unit 230 that give a characteristic grade according to a user input value and/or a similar value to a repeatedly input value; and the patent data characteristic grade classification unit 240 that gives a characteristic grade according to the number of family countries, a patent application year, and/or the number of citations.

The paper or report data characteristic grade classification unit 210 may classify the papers or reports by qualitative grades or publication years thereof, or give a weight to each of the papers or reports according to the number of citations and classify the papers or reports into final grades.

The laboratory data characteristic grade classification unit 220 may give a characteristic grade according to a user input value and/or a similarity to a repeatedly input value. For example, when the user gives data as a high reliability value or a repeatedly input value, a reliability may be set to a high value, a grade of data having a similar value to the high value may be upgraded, a grade may be lowered or a difference between grades may be increased when there is a greater difference.

The factory production data characteristic grade classification unit 230 may give a characteristic grade according to a user input value and/or a similarity to a repeatedly input value. For example, when the user gives data as a high reliability value or a repeatedly input value, a reliability may be set to a high value, a grade of data having a similar value to the high value may be upgraded, a grade may be lowered or a difference between grades may be increased when there is a greater difference.

The patent data characteristic grade classification unit 240 may give a higher grade to a patent having a greater number of family countries or a patent application has been filed more lately.

The input grade classification unit 200 may further include a weight giving unit 250 that determines an input grade in consideration of a weight and an error range according to the order of priority and/or a data collection path among a plurality of property values input by the user.

The weight giving unit 250 may determine a grade in consideration of a property weight and an error range according to the order of priority among the plurality of properties input by the user.

For example, when the user gives a priority to elastic modulus between elastic modulus and tensile strength, data in which an error range of tensile strength is smaller than an error range of elastic modulus may be classified into a higher grade.

Also, an interval between tensile strength grades having a low priority may be classified to be smaller than an interval between elastic modulus grades having a high priority.

The weight giving unit 250 may be preset by the user to give a priority to any one or more of laboratory data, factory production data, and pre-generated model data on the basis of a data collection path, thereby determining a grade in consideration of a property weight and an error range accordingly. A priority based on a collection path may be preset or changed by the user.

The weight giving unit 250 may additionally give a weight for each specific grade, give a weight for determining an input grade, and determine a final input grade in consideration of all property priorities, error ranges, and characteristic grades.

A machine learning classification algorithm used herein may be a classification algorithm or a regression algorithm belonging to supervised learning among machine learning algorithms. For example, a decision tree, a k-NN, an SVM, etc. may be used, but the scope of the present invention is not limited thereto. When there is a small number of data batches, the classification operation may be omitted as necessary. As a regression algorithm, multiple linear regression, logistic regression, SVM, deep learning, etc. may be used.

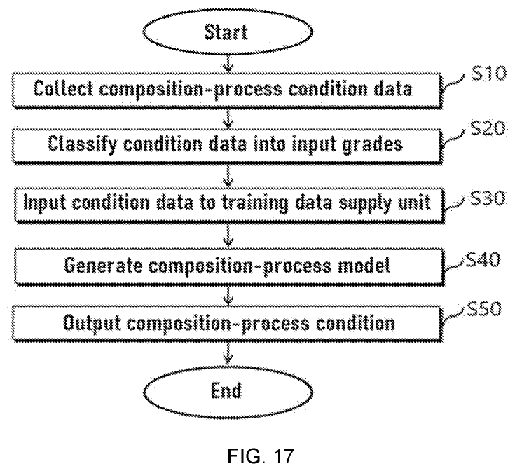

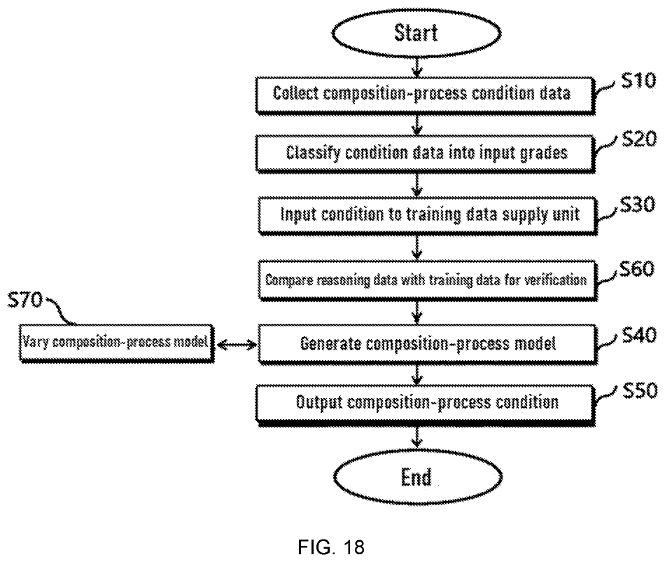

FIGS. 17 and 18 are flowcharts illustrating an operating method of a device according to an embodiment of the present invention.

Referring to FIG. 17, an AI-based method of recommending a composition-process of a composite material may include an operation S10 of collecting composition-process condition data for a target property input by a user; an operation S20 of classifying the collected condition data into different input grades according to input grade determination factors; an operation S30 of storing the condition data classified into the input grades in the training database and inputting condition data of a preset grade, specifically, a high grade or a specific grade, to the training data supply unit 300; an operation S40 of learning and verifying data input from the training data supply unit 300 to generate a composition-process model; and an operation S50 of deriving one or more composition-process conditions for the target property by the model and storing the conditions in an output database by the data output unit 500.

Referring to FIG. 18, the operation S40 of generating the composition-process model may further include an operation S60 of comparing and verifying data extracted for the target property from the reasoning database stored in the data reasoning unit 600 and the data supplied from the training data supply unit 300.

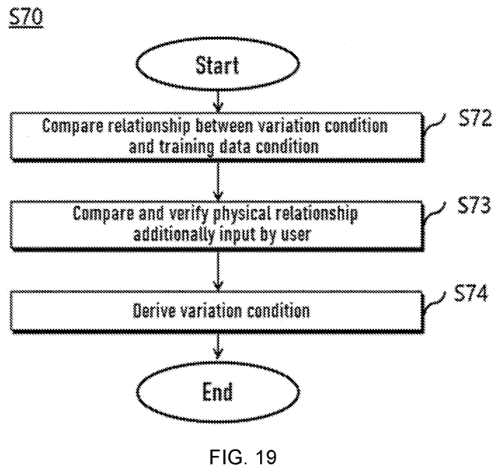

FIG. 19 is a flowchart illustrating a model variation operation method of a device according to an embodiment of the present invention.

Referring to FIG. 19, the operation S40 of generating the composition-process model may further include a model variation operation S70 of varying the model according to a variation condition input by the user.

In an embodiment of the present invention, the model variation operation S70 may include an operation S72 of comparing a relationship between a variant material condition or variant synthesis method input by the user and a material condition or synthesis method input by the training data supply unit 300; and an operation S74 of deriving a modification condition to replace one or more factors of a material composition condition and synthesis process condition generated by the model generation unit with other factors or include one or more additional factors.

In an embodiment of the present invention, the operation S72 of comparing the relationship may include an operation S73 of comparing and verifying a physical relationship additionally input by the user.

In an embodiment of the present invention, the operation S50 in which the model stores the derived composition-process conditions in the material-process output database may include an operation of storing one or more composition-process conditions generated by the model unit in a first material-process output database; an operation of classifying the conditions into separate grades according to an output grade determination factor in the output grade classification unit; and an operation of storing an output grade according to a composition-process condition satisfying an output grade determination factor input by the user in the second material-process output database.

According to an embodiment of the present invention, in the operation in which the model stores the derived composition-process conditions in the material-process output database, the output grade determination factor may be a unit price, a yield, a processing time, or a preferred process.

According to an embodiment of the present invention, in the operation of classifying the collected condition data into the different input grades, input grade determination factors may include one or more of a data type, a data characteristic, a property weight, and an error range.

According to an embodiment of the present invention, in the operation of classifying the collected condition data into the different input grades, the data type may be one or more of paper or report data, laboratory data, factory production data, and patent data.

According to an embodiment of the present invention, in the operation S20 of classifying the collected condition data into the different input grades, the paper or report data may give a weight to each of qualitative grades of academic journals, each of qualitative grades of papers or reports, and/or each of publication years of the papers or reports and give a characteristic grade according to the weight, the laboratory data and the factory production data may give a characteristic grade according to a user input value and/or a similarity to a repeatedly input value, and the patent data may give a characteristic grade according to the number of family countries, a patent application year, and/or the number of citations.

According to an embodiment of the present invention, in the operation S20 of classifying the collected condition data into the different input grades, a weight for determining an input grade may be given by considering property weights and error ranges according to the order of priority and/or a collection path among a plurality of properties input by the user and giving a weight for each specific grade. A specific example has been described above.

In an embodiment of the present invention, when the user gives a high reliability value to the laboratory data and the factory production data or when the laboratory data and the factory production data is a repeatedly input value, a reliability may be set to a high value, a grade of data having a similar value to the high value may be upgraded, a grade may be lowered or a difference between grades may be increased when there is a greater difference, and a higher grade may be given to the patent data when there are a greater number of family countries or a patent application has been filed more lately.

In an embodiment of the present invention, in the operation S20 of classifying the collected condition data into the different input grades, a weight may be given in consideration of a priority and an error range among a plurality of property values input by the user.

As an embodiment of the present invention, a computer-readable recording medium on which a program for implementing the above-described method is recorded may be provided.

In other words, the above-described method can be created as a program which is executable in a computer and implemented in a general-purpose digital computer that operates the program using a computer-readable medium. The structure of data used in the above-described method may be recorded into a computer readable medium in various ways. The computer-readable medium may be any available medium that can be accessed by a computer and encompasses all of volatile and non-volatile media and detachable and non-detachable media. Also, the computer-readable medium may encompass all computer storage media. The computer-storage media include all volatile and non-volatile media and detachable and non-detachable media that are implemented by any method or technology for storing information such as computer-readable commands, data structures, program modules, or other pieces of data. However, it should not be understood that the recording medium for recording an executable computer program or code for performing various methods of the present invention includes temporary media such as carrier waves or signals. The computer-readable medium may include storage media such as magnetic storage media (e.g., a ROM, a floppy disk, a hard disk) and optical media (e.g., a compact disc ROM (CD-ROM) and a digital versatile disc (DVD)).

Those of ordinary skill in the art should understand that the present invention can be implemented in a specific different form without changing the technical spirit or necessary characteristics of the present invention. Therefore, it should be understood that the above-described embodiments are illustrative in all aspects and are not limitative. The scope of the present invention is defined by the following claims rather than the detailed descriptions, and it should be interpreted that the meanings and scope of the claims and all changes or modifications derived from the equivalents thereto fall within the scope of the present invention.

1. An artificial intelligence-based device for analyzing a material from an unknown sample, the device comprising:
a material analysis unit configured to acquire at least three pieces of characteristic analysis data from an unknown sample;
a sample classification unit configured to compare the acquired characteristic analysis data with data included in a property database to determine a similarity score and a confidence score, and to store the scores in a sample database;
a comprehensive analysis unit configured to perform learning and verification on the basis of the similarity score and the confidence score according to a predetermined condition to analyze a material; and
a material output unit configured to store a material analysis result in an output database.

2. The device of claim 1, wherein the material output unit further comprises an output adjustment unit configured to determine an output form according to a number of estimated samples, a similarity score, or a confidence score set by a user.

3. The device of claim 1, wherein the output database comprises:
a first material output database configured to store the material analysis result of the comprehensive analysis unit; and
a second material output database configured to store a material result determined by an output adjustment unit.

4. The device of claim 1, wherein the comprehensive analysis unit gives a weight to a similarity score and a confidence score of each piece of the characteristic analysis data and analyzes the material.

5. The device of claim 1, wherein the characteristic analysis data is chemical structure data, optical property data, mechanical property data, electrical property data, thermal property data, or magnetic property data.

6. The device of claim 5, wherein the chemical structure data is at least one of nuclear magnetic resonance (NMR) data, X-ray photoelectron spectroscopy (XPS) data, energy dispersive X-ray spectroscopy (EDS) data, elemental analysis data, gel permeation chromatography (GPC) data, and cyclic voltammetry (CV) data, for which weights are preset,
the optical property data is at least one of ultraviolet/visible spectroscopy (UV-Vis) data, Fourier-transform infrared spectroscopy (FTIR) data, Raman spectroscopy data, X-ray spectroscopy (XRF) data, gamma spectroscopy data, and ellipsometry data, for which weights are preset,
the mechanical property data is at least one of universal testing machine (UTM) data, Izod impact strength data, and dynamic mechanical test analysis test data, for which weights are preset,
the electrical property data is at least one of electrical conductivity data, dielectric constant hysteresis data, and ultraviolet photoelectron spectroscopy (UPS) data, for which weights are preset,
the thermal property data is at least one of differential scanning calorimetry (DSC) data, thermogravimetric analysis (TGA) data, and thermal conductivity-T plot data, for which weights are preset, and
the magnetic property data is at least one of electron spin resonance (ESR) data and magnetoresistance analysis data, for which weights are preset.

7. The device of claim 1, wherein the property database is connected to an external database through an Internet network or a self-established network.

8. An artificial intelligence-based device for recommending a composition-process of a composite material, which comprises the device of claim 1 and outputs one or more composite-process conditions for a target property.

9. The device of claim 8, comprising:
a data collection unit configured to collect composition-process condition data for a target property input by a user and store the collected condition data in a collection database;
an input grade classification unit configured to classify the collected condition data into different input grades according to input grade determination factors;
a training data supply unit configured to store the condition data classified into the input grades in a training database and input condition data of a preset high grade in the training database;
a model generation unit configured to learn and verify the data input from the training data supply unit and generate a composition-process model; and
a material-process data output unit configured to derive one or more composition-process conditions for the target property and store the derived composition-process conditions in a material-process output database.

10. The device of claim 9, further comprising:
a data reasoning unit configured to extract reasoning condition data for the target property and send the extracted reasoning condition data to the model generation unit such that the reasoning condition data is verified by being compared with the condition data supplied from the training data supply unit; and
a model variation unit configured to vary the model generated by the model generation unit according to a variation condition input by the user.

11. An artificial intelligence-based method of analyzing a material from an unknown sample, which uses the device of claim 1 and is implemented by a computer, the method comprising:
acquiring at least three pieces of characteristic analysis data from an unknown sample;
comparing the acquired characteristic analysis data with the data included in the property database to determine a similarity score and a confidence score; and
performing learning and verification on the basis of the similarity score and the confidence score according to a predetermined condition to analyze a material and storing a material analysis result in an output database.

12. An artificial intelligence-based method of recommending a composition-process of a composite material, which uses the device of claim 8 and is performed by an artificial intelligence-based device implemented by a computer for recommending a composition-process of a composite material, the method comprising:
collecting composition-process condition data for a target property input by a user;
classifying the collected condition data into different input grades according to input grade determination factors;
storing the condition data classified into the input grades in a training database and inputting condition data of a predetermined high grade to a training data supply unit;
learning and verifying the data input from the training data supply unit to generate a composition-process model; and
deriving, by the model, one or more composition-process conditions for the target property and storing, by a material-process data output unit, the derived composition-process conditions in a material-process output database.

13. The method of claim 12, wherein the generation of the composition-process model further comprises:
verifying data derived for the target property from a reasoning database stored in a data reasoning unit by comparing the data derived for the target property with the data supplied from the training data supply unit; and
a model variation operation of varying the model according to a variation condition input by the user.
